(19) United States
(12) Patent Application Publication
(10) Pub. No.: US 2025/0387178 A1
(43) Pub. Date: Dec. 25, 2025

(54) INTELLIGENT UTILIZATION OF SURGICAL ROBOTIC INSTRUMENTS AND MANIPULATORS

(71) Applicant: INTUITIVE SURGICAL OPERATIONS, INC., Sunnyvale, CA (US)

(72) Inventors: Omid Mohareri, San Francisco, CA (US); Muhammad Abdullah Jamal, Sunnyvale, CA (US)

(21) Appl. No.: 19/244,755

(22) Filed: Jun. 20, 2025

Related U.S. Application Data
(60) Provisional application No. 63/663,566, filed on Jun. 24, 2024.

(51) Int. Cl. A61B 34/30; A61B 34/10; A61B 34/20; G16H 20/40

(52) U.S. Cl. CPC A61B 34/30; A61B 34/10; A61B 34/20; G16H 20/40

(57) ABSTRACT

Systems and methods are described for determining task based utilization of instruments and repositionable structures. The system may include one or more repositionable structures operatively coupled to one or more instruments, and a control system operably coupled to the one or more repositionable structures, the control system configured to receive a plurality of data streams from one or more data sources and analyze the data streams to identify a task to be performed; determine, based on the task to be performed and via an actor selection machine learning model, one or more selected instruments for performing the task, or a selected repositionable structure of the repositionable structures; generate, via a robotic action machine learning model, one or more action tokens for controlling the repositionable structures based on the task and the selected instrument or selected repositionable structure; and control the selected instrument or selected repositionable structure to perform the task.

### CROSS-REFERENCE TO RELATED APPLICATIONS

This application claims priority to and the benefit of the filing date of provisional U.S. Patent Application No. 63/663,566 entitled “INTELLIGENT UTILIZATION OF SURGICAL ROBOTIC INSTRUMENT AND MANIPULATORS,” filed on Jun. 24, 2024. The entire contents of the provisional application are hereby expressly incorporated herein by reference.

### TECHNICAL FIELD

The present disclosure relates generally to computer-assisted systems and more particularly to training and utilizing artificial intelligence to manage the utilization of robotic manipulators and instruments for performing tasks.

### BACKGROUND

Computer-assisted manipulator systems (“manipulator systems”), sometimes referred to as robotically assisted systems or robotic systems, may include one or more manipulators that can be operated with the assistance of an electronic controller (e.g., computer or control system) to move and control functions of one or more instruments coupled to the manipulators. A manipulator generally includes mechanical links connected by joints. An instrument is removably (or permanently) coupled to one of the links, typically a distal link of the plural links. In some embodiments, manipulator systems are used in conjunction with one or more auxiliary devices (e.g., a surgical bed, an insufflator, etc.).

Typically, the electronic controller must consider many different types of data in controlling the manipulator system. For example, an endoscope may provide endoscopic images or video to the controller and the controller may perform image processing or machine vision processes to determine specific movements or for controlling the manipulator system to perform specific tasks. While endoscopic images are just one example of data considered in controlling a manipulator system, many such systems involve analysis of multiple data streams to determine appropriate tasks to complete a procedure. Accordingly, it may be beneficial to implement machine learning models to assist in the processing of the data included in the data streams to improve the task generation process.

A Robotic Transformer (RT) model is one machine learning model architecture that is configured to implement automated controls. A drawback with RT models is that the inference time generally increases quadratically with each input parameter in the model feature space. Accordingly, as additional data streams are ingested into the model and are associated with corresponding parameters, control systems for manipulator systems are unable to perform the inference in real-time (e.g., within 5 ms, 10 ms, 15 ms, etc.). That is, to the high processing demands and data bandwidths required to obtain, embed, and process all of the received different data modalities (e.g., multiple sources of images or video, diagnostic sensor data, pressure sensor data, audio data, robotic system data (including kinematic data, force sensing data, event data), etc.) results in processing times that exceed what is required to safely implement RT models in a closed-loop control system. Accordingly, closed-loop, autonomous robotic manipulation using RT (or other similar models) are not currently practical for use in surgical environments.

Accordingly, there is a need for improved techniques that enable semi-autonomous, and fully autonomous closed-loop control of robotic manipulation systems. Such techniques can allow improved surgical outcomes, improved task workflows, and reduced reliance on highly skilled practitioners with niche skillsets who may otherwise not be available for an operation or procedure. These improvements further allow for wider access to surgical treatment and diagnosis across a broad range of medical and clinical domains.

### SUMMARY

The following presents a simplified summary of various examples described herein and is not intended to identify key or critical elements or to delineate the scope of the claims.

In some aspects, the techniques described herein relate to a computer-assisted system, the system including: one or more repositionable structures operatively coupled to one or more instruments; and a control system operably coupled to the one or more repositionable structures, wherein the control system is configured to: receive a plurality of data streams from one or more data sources; analyze one or more data streams from the plurality of data streams to identify a task to be performed by the one or more repositionable structures or the one or more instruments; determine, based on the task to be performed and via an actor selection machine learning model, at least one of (i) one or more selected instruments for performing the task, or (ii) a selected repositionable structure of the one or more repositionable structures for performing the task; and generate, via a robotic action machine learning model, one or more action tokens for controlling the at least one of the selected instruments or the selected repositionable structures; and control the at least one of the selected instruments or the selected repositionable structures to perform the task based upon the one or more generated action tokens.

In some aspects, the techniques described herein relate to a method for performing automated surgical tasks via a computer-assisted system including one or more repositionable structures operatively coupled to respective instruments, and a control system operatively coupled to the one or more repositionable structures, the method including: receiving a plurality of data streams from one or more data sources; analyzing the data streams to identify a task to be performed by the one or more repositionable structures; determining, based on the task to be performed and via an actor selection machine learning model, at least one of (i) one or more selected instruments for performing the task, and (ii) one or more selected repositionable structures of the one or more repositionable structures for performing the task; and generating, via a robotic action machine learning model, one or more action tokens for controlling the at least one of the determined selected instruments or the selected repositionable structures; and controlling the at least one of the determined selected instruments or selected repositionable structures to perform the task based upon the generated action tokens.

In some aspects, the techniques described herein relate to a computer-assisted system for performing automated tasks, the system including: one or more repositionable structures configured operatively coupled to respective instruments; and a control system operably coupled to the repositionable structure, wherein the control system is configured to: receive a plurality of data streams from one or more data sources; analyze one or more data streams from the plurality of data streams to identify one or more tasks to be performed by the one or more repositionable structures; input embeddings of the one or more tasks and at least one of the plurality of data streams into a robotic action machine learning model to generate one or more action tokens for controlling the one or more repositionable structures; determine a selected repositionable structure or selected instrument to implement the action token; convert the action token to a control command adapted to the selected repositionable structure or instrument; and control the selected repositionable structure or instrument based upon the control command.

In some aspects, the techniques described herein relate to a method for performing automated surgical tasks via a computer-assisted system including one or more repositionable structures operatively coupled to respective instruments, and a control system operatively coupled to the one or more repositionable structures, the method including: receiving a plurality of data streams from one or more data sources; analyzing the data streams to identify one or more tasks to be performed by the one or more repositionable structures; inputting embeddings of the one or more tasks and at least one of the plurality of data streams for into a robotic action machine learning model to generate one or more action tokens for controlling the one or more repositionable structures; determining a selected repositionable structure or selected instrument to implement the action token; converting the action token to a control command adapted to the selected repositionable structure or selected instrument; and controlling the selected repositionable structure or selected instrument based upon the control command.

In some aspects, the techniques described herein relate to a computer-readable media storing instructions that, when executed by a control system of a computer-assisted system, causes the computer-assisted system to perform any of the methods described herein.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a diagram of a computer-assisted system in accordance with one or more embodiments.

FIG. 2 is a schematic diagram of a system for determining task specific data streams for controlling a repositionable structure.

FIG. 3 is a schematic diagram of a robotic action module for generating actions tokens to control a repositionable structure.

FIG. 4 is a schematic diagram of an example task generation module for determining task specific data streams for controlling a repositionable structure.

FIG. 5 is a schematic diagram of an example task selection module for determining task specific data streams for controlling a repositionable structure.

FIG. 6 is a schematic diagram of an example data modality selection module for determining task specific data streams for controlling a repositionable structure.

FIG. 7 is a schematic diagram of an example instrument/arm/auxiliary device selection module for determining instruments, arms, and/or auxiliary devices for performing specific tasks.

FIG. 8A is a schematic diagram illustrating a process for detokenizing action tokens.

FIGS. 8B and 8C depict de-tokenized commands for controlling a repositionable structure.

FIG. 9 is a flow diagram of a method for determining task specific data streams for controlling a repositionable structure.

FIG. 10 is a flow diagram of a method for determining task specific instruments and manipulator arms for controlling.

FIG. 11 is a flow diagram of a method for generating task specific action tokens and determining specific instruments and manipulator arms for controlling a repositionable structure.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe the relation of one element or feature to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. A device may be otherwise oriented and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. Additionally, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, system, or module may, whenever practical, be included in other embodiments, implementations, systems, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

This disclosure occasionally refers to the disclosed techniques being applied to “patients” undergoing a “medical procedure.” It should be appreciated that these references are not intended to limit the application of the disclosed techniques to applied medicine contexts. For example, the described techniques can be applied to facilitate physician training, equipment testing and/or calibration, and/or other contexts. Accordingly, any reference to the term “patient” is done for ease of explanation and also envisions the application of the described techniques to a generic “subject.”

The word “task” is used herein to refer to a discrete portion of procedure that may be autonomously, semi-autonomously, or manually implemented in furtherance of a procedure. For example, a task may be to move an endoscope to a particular portion, to advance an instrument to a particular depth, to replace an instrument coupled to a manipulator, and so on. In some embodiments, a task is associated with component tasks to accomplish an overall goal. For example, a task to analyze a worksite may include component tasks related to moving an endoscope to view the worksite, advancing an instrument to predetermined depth, and enabling a functionality supported by the instrument.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

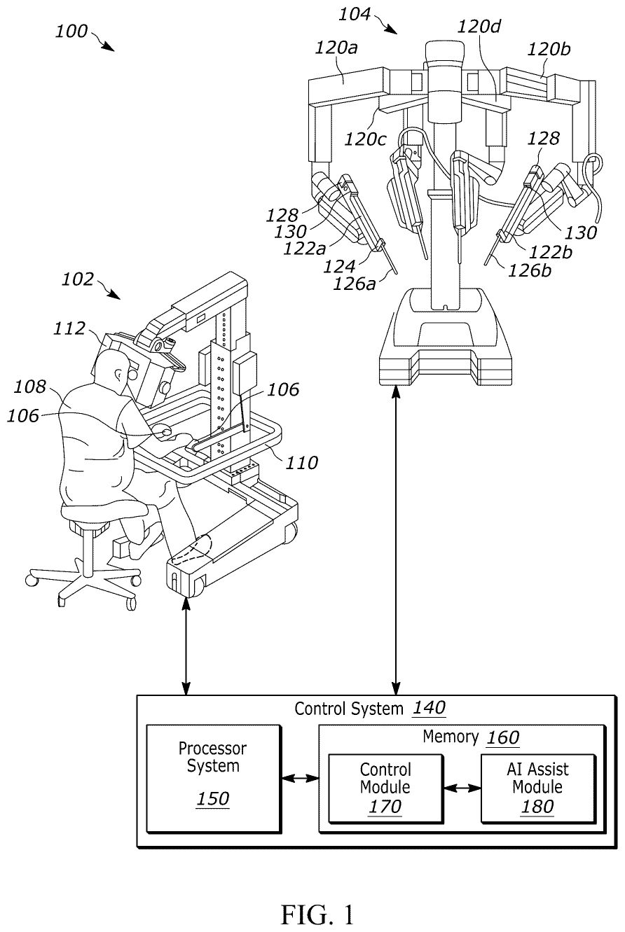

FIG. 1 is a simplified diagram of an example computer-assisted system 100, according to various embodiments. In some examples, the computer-assisted system 100 is a teleoperated system. In medical examples, the computer-assisted system 100 can be a teleoperated medical system such as a surgical system. As shown, the computer-assisted system 100 includes a follower device 104 that can be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include the workstation 102.

In the example of FIG. 1, the workstation 102 includes one or more leader input devices 106 that are designed to be contacted and manipulated by an operator 108. For example, the workstation 102 may comprise one or more leader input devices 106 for use by the hands, the head, or some other body part(s) of operator 108. The leader input devices 106 in this example are supported by the workstation 102 and can be mechanically grounded. In some embodiments, an ergonomic support 110 (e.g., forearm rest) can be provided on which the operator 108 can rest his or her forearms. In some examples, the operator 108 can perform tasks at a worksite within a workspace near the follower device 104 during a procedure, by commanding the follower device 104 using the leader input devices 106. In a medical example, the worksite may be a surgical worksite associated with a patient.

A display device 112 is also included in the workstation 102. The display device 112 may be configured to display images for viewing by the operator 108. The display device 112 can be moved in various DOFs to accommodate the viewing position of the operator 108 and/or to provide control functions. In embodiments where the display device 112 provides control functions, the leader input devices 106 may include the display device 112. In the example of the computer-assisted system 100, displayed images may depict a worksite at which the operator 108 is performing various tasks by manipulating the leader input devices 106 and/or the display device 112. In some examples, images displayed by display device 112 may be received by the workstation 102 from one or more imaging devices arranged at a worksite. In other examples, the images displayed by the display device 112 may be generated by the display device 112 (or by a different connected device or system), such as for virtual representations of tools, the worksite, or for user interface components. As will be explained below, in some embodiments the display device 112 may display one or more tasks for the operator 108 to perform with respect to any component of the computer-assisted system 100.

As illustrated, the computer-assisted system 100 also includes a follower device 104 that can be commanded by the workstation 102. In a medical example, the follower device 104 can be located near an operating table (e.g., a table, bed, or other support) on which a patient can be positioned. In some medical examples, the workspace is provided on an operating table, e.g., on or in a patient, simulated patient, or model, training dummy, etc. (not shown). As illustrated, the follower device 104 may include a plurality of repositionable structures 120 (sometimes referred to as “manipulator arms” in robotic embodiments). In some embodiments, the repositionable structures 120 may include a plurality of links that are rigid members and joints that can be individually actuated as part of a kinematic series. Additionally, each of the repositionable structures 120 is configured to couple to an instrument 122. While FIG. 1 illustrates a follower device 104 that has four repositionable structures 120a-120d, in other embodiments, the follower device 104 may include one, two, three, four, five, six, or additional or fewer repositionable structures 120a-120d.

The instrument 122 can include, for example, a working portion 126 and one or more structures for supporting and/or driving the working portion 126. Example working portions 126 include end effectors that physically contact or manipulate material, energy application elements that apply electrical, RF, ultrasonic, or other types of energy, sensors that detect characteristics of the workspace environment (such as temperature sensors, imaging devices, etc.), and the like. In various embodiments, examples of instruments 122 include, without limitation, a sealing instrument, a cutting instrument, a sealing-and-cutting instrument, an energy instrument for applying energy, a gripping instrument (e.g., clamps, jaws), a stapler, an imaging instrument such as one using optical, RF, or ultrasonic imaging modalities, a sensing instrument, an irrigation instrument, a suction instrument, and/or the like. In addition, the instrument 122 may include a transmission mechanism 128 that can be coupled to a drive assembly 130 of the respective repositionable structure 120a-120d. The drive assembly 130 may include a drive and/or other mechanisms controllable from workstation 102 that transmit forces to the transmission mechanism 128 to articular or otherwise actuate the instrument 122.

As illustrated, each instrument 122 may be mounted to a portion of a respective repositionable structure 120a-120d. In FIG. 1, this is shown with the drive assembly 130 physically coupled to the transmission mechanism 128. The distal portion of each repositionable structure 120a-120d further includes a cannula mount 124 to which a cannula (not shown) is mounted. When a cannula is mounted to the cannula mount 124, a shaft of the instrument 122 passes through the cannula and into a workspace.

In various embodiments, one or more of the working portions 126 of the instruments 122 may include an imaging device for capturing images. The imaging device may include any sensing technology capable of acquiring an image. Example imaging instruments include an optical endoscope, a hyperspectral camera, an ultrasonic sensor, etc. Imaging instruments may comprise monoscopic imagers, stereoscopic imagers, and/or the like. Imaging devices based on radiofrequency domains may capture images in any frequency spectrum, including visible light, infrared light, ultraviolet light, and/or the like. The imaging device may include an illumination source to light the region being imaged. In embodiments where the working portions 126 of one or more of the instruments 122 include an imaging device, the instrument 122 may be configured to capture images of a portion of the workspace for display via the display device 112.

In some embodiments, the repositionable structures 120a-120d and/or instruments 122 can be controlled to move the working portion 126 in response to manipulation of the leader input devices 106 by the operator 108. Accordingly, the repositionable structures 120a-120d and/or instruments 122 may be said to “follow” the leader input devices 106 through teleoperation. This enables the operator 108 to perform tasks at the worksite using the repositionable structures 120a-120d and/or instruments 122. For a surgical example, the operator 108 can direct the repositionable structures 120a-120d of the follower device 104 to move the working portions 126 as part of a surgical procedure performed at an internal surgical site that is entered via one or more minimally invasive apertures or natural orifices. It should be appreciated that, in some embodiments, the follower device 104 may include non-teleoperated components that the operator 108 or other medical professional must manually manipulate to a desired pose.

In some embodiments, a repositionable structure 120a of the computer-assisted system 100 may be configured to support a working portion 126a that includes an imaging device (also referred to herein as an “imaging device 126a”). For convenience, an instrument 122 that includes an imaging device is also referred to as an “imaging instrument” herein. The control system 140 may be configured to command the repositionable structure 120a and/or the imaging instrument 122 comprising the imaging device 126a to automatically position and/or orient (“pose”) the field of view (FOV) of the imaging device 126a to provide images of the workspace and/or other instruments 122.

In the illustrated embodiment, a control system 140 is communicatively coupled to the workstation 102. In other embodiments, the control system 140 may be provided as a component of the workstation 102 and/or the follower device 104. During teleoperation, as the operator 108 moves the leader input device(s) 106, one or more sensors configured to detect the leader input device(s) 106 generate spatial and/or orientation movement data that is provided to control system 140. The control system 140 may interpret the spatial and/or orientation information to determine and/or provide control signals to the follower device 104 to control the movement of repositionable structures 120a-120d, instruments 122, and/or working portions 126. In addition to the components of the follower device 104, in some embodiments, the control system 140 is configured to interpret inputs received from the workstation 102 to control operation of one or more auxiliary devices (not depicted) utilized in a procedure. For example, the workstation 102 may be used to control a pose of a surgical bed or operation of an insufflator.

In one embodiment, the control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like) for communications between the control system 140 and the workstation 102 and/or the follower device 104.

In some embodiments, the control system 140 may be implemented at one or more computing systems. For example, one or more computing systems may be used to control the follower device 104. As another example, one or more computing systems may be used to control components of the workstation 102, such as movement of a display device 112.

As illustrated, the control system 140 includes a processor system 150, a memory 160, and an artificial intelligent (AI) assist module 180. The memory 160 may store a control module 170. The processor system 150 may include one or more processors having different processing architectures for processing instructions. For example, the one or more processors may be one or more cores or micro-cores of a multi-core processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like.

In some embodiments, the processor system 150 includes circuitry to support one or more communication interfaces (e.g., Bluetooth interface, infrared interface, network interface, optical interface, etc.). Additionally, a communication interface of control system 140 may include an integrated circuit for connecting the control system 140 to a network (not shown) (e.g., a local area network (LAN), a wide area network (WAN) such as the Internet, mobile network, or any other type of network) and/or to another device, such as the workstation 102 and/or the follower device 104.

Additionally, the memory 160 may include non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, and/or any other memory chip or cartridge. The non-persistent storage and persistent storage are examples of non-transitory, tangible machine-readable media that can store executable code that, when run by one or more processors (e.g., processor system 150), can cause the one or more processors to perform one or more of the techniques and/or methods disclosed herein.

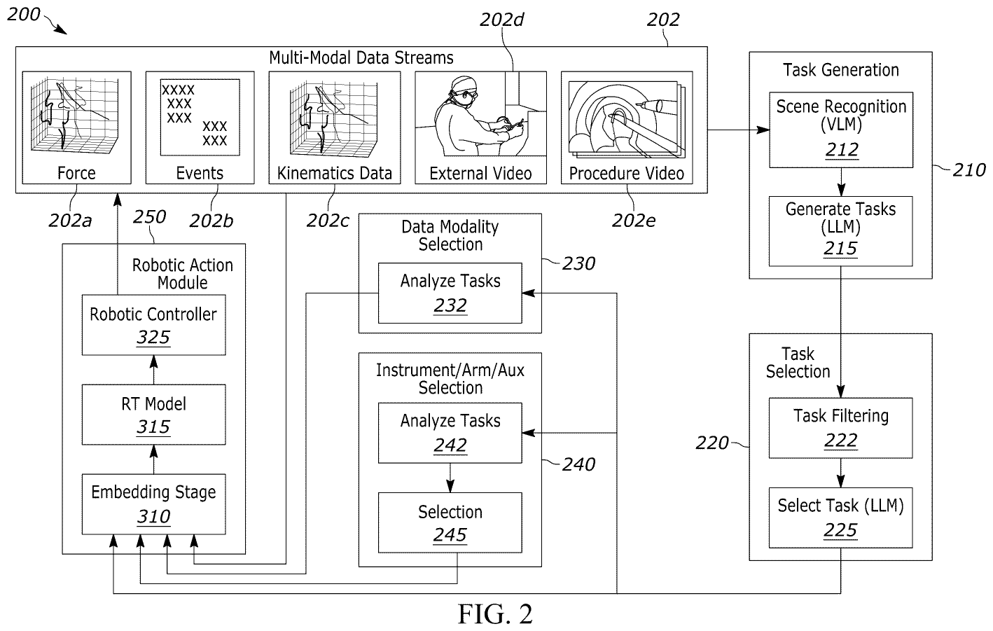

The AI assist module 180 may implement one or more machine learning models and/or training protocols therefor. For example, the AI assist module 180 may implement one or more neural networks, deep learning models, decision trees, support vector machines, linear regression, generative AI models, reinforced learning models, random forests, Naïve Bayes models, large language models (LLMs), generative adversarial networks, foundation models, image recognition models, linear discriminant analysis models, creative applications, autoregressive models, supervised or unsupervised learning models, multimodal models, vision language models (VLMs), vision foundation models (VFMs), large multi-modal models (LMMs), Transformer models (including Robotic Transformer models), or another machine learning or AI model for performing the methods described herein. The structure of the one or more machine learning is described in more detail with respect to FIGS. 2-7. The AI assist module 180 may include dedicated processors and memory for storing and performing AI processes, or the AI assist module 180 may utilize resources of the processor system 150 and the memory 160 to store and/or perform any processing or tasks required to perform the methods described herein.

Additionally, the control system 140 may also include one or more input devices (such as a touchscreen, keyboard, mouse, microphone, touchpad, electronic pen, or any other type of input device) and/or output devices (such as a display device, a speaker, external storage, a printer, or any other output device). In some embodiments, the control system 140 may be implemented on a particular node of a distributed computing system (e.g., a cloud computing system). As another example, different functionalities associated with the control system 140 may be implemented on different nodes of the distributed computing system. Further, one or more elements of the aforementioned control system 140 may be located at a remote location and connected to the other elements over a network.

In an endoscopic surgery example, the imaging instrument comprising the imaging device 126a may be inserted into the patient prior to the other instruments 122, including a second instrument 122b comprising a second working portion 126b. The second instrument 122b can include any appropriate working portion 126b, and can even include a second imaging device. Accordingly, the imaging device 126a may be maneuvered to positioned to identify a target to which other instruments may interact with as part of another task. The control system 140 may, for example, automatically command the corresponding repositionable structures 120a and 120b to position respective instruments 122a and 122b to perform one or more tasks in tandem, or sequentially based on the specific task, instruments, and positions of the repositionable structures 120a and 120b. In examples described further herein, the control system 140 may further determine specific types of instruments and specific arms of the repositionable structures 120 to optimize the completion of a given task. Further, the control system 140 may perform AI processes and algorithms via the AI assist module 180 to determine specific data stream modalities required to perform a given task either automatically via the system 100, or with user assistance or input to the system 100. Further, the controls system 140 may determine a task that is to be performed entirely manually by an operator such as by the operator 108, and the control system 140 may further determine the specific data stream modalities to provide to the operator 108 in performing the task.

The disclosed techniques enable the computer-assisted system 100 to perform real-time operations and tasks in surgical settings. The computer-assisted system 100 can automatically perform the tasks by only embedding determined task-specific data modalities when generating inputs into a robotics transformer (RT) model. Typically, Robotics Transformers models are not capable of real-time control of complex robotic systems, such as computer-assisted systems used to perform a surgical procedure, due to the high processing demands and data bandwidths required to obtain, embed, and process required all of the supported data modalities. The disclosed systems and methods dynamically adjust the input data modalities based on the particular task being performed. Thus, only a subset of the data modalities is embedded at a given time. This significantly reduces the number of input tokens to the RT model. As a result, the control system of a computer-assisted system is able to generate control commands using the RT model in real-time, thereby enabling the various efficiencies provided by performing closed-loop control using a Robotics Transformer. Such techniques can also improve the efficiency of operating the computer-assisted system or instrument, simplify user control of the computer-assisted system or instrument, improve the accuracy of a procedure or tasks require for a procedure. Further, although a surgical example is shown, the disclosed techniques provide an improvement to the computer-assisted system 100 in the non-surgical aspects of the procedure, and can be used to improve computer-assisted systems applied in non-medical contexts.

FIG. 2 is a schematic diagram of a system 200 for determining task specific data streams for controlling a repositionable structure (such as the repositionable structure 120). The system includes a series of modules that may be executed by the control system 140 (e.g., via the AI assist module 180 of FIG. 1) to control the repositionable structure to perform one or more tasks. The system 200 includes sources of multi-modal data including one or more multi-modal data streams 202, a task generation module 210, a task selection module 220, a data modality select module 230, an instrument/arm select module 240, and a robotic action module 250. It should be appreciated that in other embodiments, additional or fewer modules may be implemented. Further, in other embodiments, one or more of the described modules may be combined into a single module.

The system 200 includes one or more sources of multi-modal data 202. For example, the multimodal data 202 may include a data stream 202a indicative of force exerted upon an instrument, a data stream 202b indicative of system events generated by the repositionable structure and/or a control system thereof, a data stream 202c indicative of kinematic data associated with the repositionable structure and/or instruments or auxiliary devices associated therewith, an external video data stream 202d, a procedure video data stream 202e (such as image data generated by an endoscope), and/or other sources of data that indicate a state of a procedure facilitated by the repositionable structures.

The system 200 may be configured to route the multimodal data 202 to a task generation module 210 configured to output one or more tasks that may be performed in furtherance of the procedure based on a procedure state represented by the input multimodal data 202. The task generation module 210 may identify tasks that are to be perform in the near term (e.g., tasks that respond to conditions detected in an input set of multi-modal data) and/or tasks that are to be performed in the long term (e.g., tasks that will need to be performed later in the procedure after one or more near-term tasks are performed). By also generating long-term tasks, the system 200 is able to track preparedness for performing the long-term task and generate additional tasks to prepare the control system for performing the long-term task as the procedure advances closer to the appropriate time for performing the long-term tasks. In some embodiments, the task generation module 210 may include component models to facilitate the analysis. For example, the task generation module 210 may include a scene recognition model 212 to parse image/video data and generate data describing what is in the scene and where it the identified objects are located within the image data included in the multimodal data 202. In the case of a visual-language model (VFM), the output may be in the format of a natural language text description. The task generation module 210 may also include a task generation model 215 configured to actually generate the one or more tasks.

The system 200 may then provide the output tasks to a task selection module 220 to select which tasks should actually be implemented by the control system and/or an operator thereof. As illustrated, the task selection module may include a task filter model 222 configured to filter the tasks generated by the task generation model 215 and a task selection model 225 configured to select one or more valid tasks to be performed by the control system and/or an operator thereof. The system 200 may then provide the selected tasks to the data modality selection module 230, instrument/arm selection module 240, and/or the robotic action module 250.

The data modality selection module 230 may be configured to analyze the one or more selected tasks to determine one or more task specific data streams needed to implement the selected tasks. As described above, each task may require a particular type of data to safely execute the task. Accordingly, the data modality selection module 230 may include a task analysis model 232 configured to identify the required data modalities for performing the selected tasks. It should be appreciated that a required data modality may include a particular data stream of the multimodal data 202 (e.g., kinematic data or procedure video) or a particular state of the multimodal data (e.g., that the procedure video is required to captured image data of a particular object, such a target anatomy). It should be appreciated if a required data stream is unavailable, the data modality selection module 230 may provide a notification to an operator indicating that a task cannot be performed due to the data modality unavailability and/or interact with the task selection module 220 to select a different task where the required data streams are all available and/or the task generation module 210 to generate a task to make the required data stream available (e.g., by enabling a sensor or by commanding a camera to reposition a field of view). The data modality selection module 230 may then provide a selection of the available data streams to the robotic action module 250.

Because the system 200 may be configured to process multiple tasks synchronously, the data modality selection module 230 may be configured to associate the selected available data streams with each task prior to providing the selection to the robotic action module 250. As a result, the robotic action module 250 may be able switch data modalities based on the particular tasks being converted into robotic action.

Like the data modality selection module 230, the instrument/arm selection module 240 may be configured to receive the one or more selected tasks from the task selection module 220. In response, the instrument/arm selection module 240 may be configured to determine a particular instrument of the one or more instruments and/or a particular arm of the one or more arms of the repositionable structures for performing the selected tasks. Accordingly, the instrument/arm selection module 240 may include a task analysis model 242 configured to identify which instruments and/or arms of the repositionable structure are capable of implementing the selected tasks and an instrument/arm selection model 245 configured to assign the task to a particular instrument/arm. For example, if the system 200 is used to control multiple arms/instruments coupled to gripper end effectors, the instrument/arm selection module 240 may identify the instrument/arm corresponding to a particular gripper end effector selected to perform the task. It should be appreciated that in some scenarios, the appropriate instrument is within the procedural theater, but not coupled to repositionable structure. Accordingly, in this scenario, the instrument/arm selection module 240 may interface with the task generation module 210 to generate one or more tasks related to swapping an instrument coupled to the arm such that the task can be performed. The instrument/arm selection module 240 then provides data indicative of specific instruments and/or arms for performing one or more of the selected tasks to the robotic action module 250. It should be appreciated that in many procedures, there is a single auxiliary device of each auxiliary device type operatively coupled with the control system. However, if there are multiple auxiliary devices of the same auxiliary device type operatively coupled with the control system, the instrument/arm selection module 240 may additionally select which auxiliary device is to implement a selected task.

In examples, the outputs of task selection module 220 and/or the instrument/arm selection module 240 may be provided to the operator, such as via the workstation 102 of FIG. 1. The operator may then approve of the tasks to be performed and/or the instrument/arm assignment prior to implementing the selected tasks. If the operator disapproves the selected tasks, the task selection module 220 may provide alternative tasks that can be performed. In some embodiments, if the operator disapproves the assignment of the tasks, the operator may be able to manually assign the task to the preferred instrument/arm via the display device 112.

As described above, the robotic action module 250 may be configured to receive the selected tasks from the task selection module 220, task specific data modalities from the data modality selection module 230, the instrument/arm assignment information from the instrument/arm selection module 240, and the multimodal data 202. The robotic action module 250 may then process the multimodal data 202 corresponding to the selected data modalities to generate action tokens to be control the instruments/arms assigned to perform the selected tasks. More particularly, the robotic action module 250 may embed the data modalities via an embedding stage 310 for input into an RT model 315. The robotic action module 250 may then convert the action tokens to command one or more components of a computer-assisted robotic device (such as the follower device 104 of FIG. 1) to perform one or more of the selected tasks utilizing one or more of the selected instrument(s), arms(s) and auxiliary device(s).

It should be appreciated that while the system 200 includes both the data modality selection module 230 and the instrument/arm selection module 240, in some embodiments, the system 200 only includes one of the modules 230, 240. Additionally, in some embodiments, one or more of the modules 210, 220, 230, 240 may be combined into a single module, for example, by implementing chain of thought (CoT) or other recurrent prompting techniques such that a single prompt performs the described functionality corresponding to the individual modules.

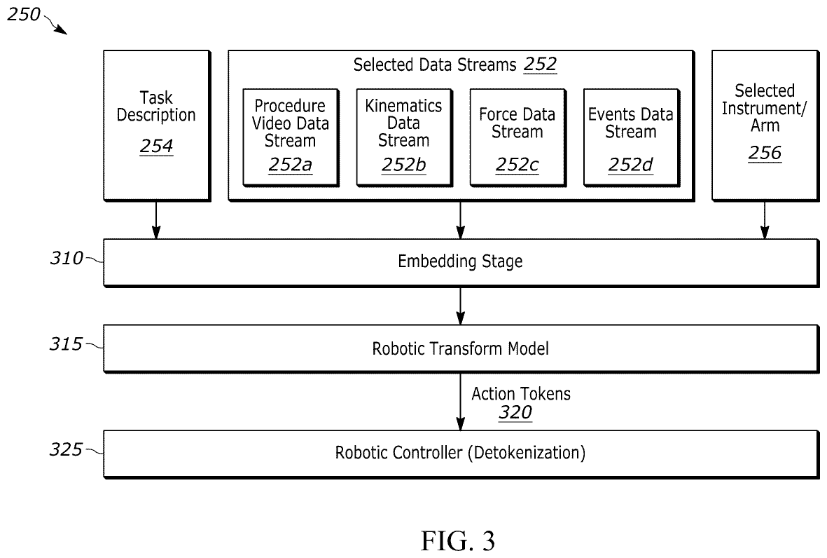

FIG. 3 is a schematic diagram of a robotic action module 250 (such as the robotic action module 255 of FIG. 2), for generating actions tokens to control a repositionable structure (such as the repositionable structures 120 of FIG. 1). The robotic action module 250 may be implemented as part of the AI assist module 180 of FIG. 1. As described with respect to FIG. 2, the robotic action module 250 may be configured to receive as inputs one or more data streams forming the multi-modal data 202, an indication of a selected task 254, and an indication of a selected instruments and/or arms 256.

As illustrated, the robotic action module 250 includes an embedding stage 310 that embeds the various data modalities (e.g., text, image, audio, video, sensor data, etc.) into a common data space for input into an RT model 315. As described above, the transform architecture inference performance is dependent on the number of tokens input into the model. Thus, to reduce the number of input tokens to the RT model 315, the robotic action module 250 nay be configured to only embed the selected data streams 252 required to perform the selected task 254. As one example, if the data modality selection module 230 indicated that endoscope data and kinematic data are the only required data streams for performing a particular task, the robotic action module 250 may only input tokens associated with the procedure video data stream 252a and the kinematic data stream 252b into the RT model 315. The robotic action module 250 may either refrain from inputting the non-required data streams into the corresponding embedding model of the embedding stage 310 or refrain from including the outputs of the corresponding embedding model of the embedding stage 310 in the prompt to the RT model 315. For example, in the latter scenario, the same embedding model may be used by both the RT model 315 and a LLM or LMM of another module disclosed herein (such as the task generation module 210). Accordingly, not every available data stream or even every embedded available data stream that form the multimodal data 202 is embedded for input into the RT model 315.

The embedding stage 310 may be configured to project the input data streams into a common vector space. In examples, the embedding stage 310 may implement one or more embedding methods including, without limitation, a word2vec, GloVe, ELMo, BERT, principal component analysis, singular value decomposition, a transformer model, Doc2Vec, paragraph vectors, convolutional neural networks, pre-trained models for image embedding, node embeddings, iknowledge graph embeddings (e.g., TransE, TrensR, DistMult, etc.), word embeddings, graph embeddings, entity embeddings, or another type of embedding supported by the RT model 315. It should be appreciated that while the embedding stage 310 is depicted as a single block, each data stream 252 may be associated with a respective embedding model for generating embeddings of the respective data type included within the embedding stage 310.

The various embedding models implemented by the embedding stage 310 may be trained and/or fine-tuned using historical data of prior procedures. As one example, an embedding model to analyze a procedure video data stream 252a may be trained to identify objects (e.g., instruments, ports, anatomical features, etc.) that are expected to be seen during the procedure. In this example, the embedding model for the procedure video data stream 252a may be trained and/or fine-tuned using historical image data in which the pixels representative of the corresponding objects are labels. The embedding model may then be trained or tuned in any suitable manner that minimizes loss with respect to the set of ground truth labels.

As another example, an embedding model for a kinematic data stream 252b, force data stream 252c, and/or an events data stream 252d may be trained using labeled segments from historical procedures performed using the same type of manipulator system associated with the system 200. For example, after a historical procedure has been performed, the set of kinematic, force, and/or event data captured during the historical procedure may be correlated, compiled, and aligned in time. For some types of conditions, the set of data may then be labeled by identifying time segments at which a particular condition occurred (e.g., an instrument or end effector being operated in a particular manner, performance of a particular phase of a procedure, transition to a new phase of a procedure, etc.). Accordingly, by building a library of labeled time segments, an embedding model can be trained to identify the conditions in the corresponding data streams when implemented in the robotic action module 250. In these embodiments, the embedding model may include a time-series analysis model (such as a Transformer model or a recurrent neural network (RNN)) configured to capture the temporal relationship between data within the labeled time segments.

For other conditions (such as alerts, errors, anomalies, abrupt and/or significant changes in operation, or other instanteous conditions), the representation of that condition in the historical data may be labeled such that the embedding model can be trained to detect the labeled conditions. In either case, the training system may implement self-supervised learning to identifying the conditions and/or fine-tuning based on the procedure type and/or manipulator system such that the identification is tailored to the procedure/manipulator system associated with the system 200.

The robotic action module 250 then provides the embedded data output from the embedding stage 310 as an input to the RT model 315. The RT model 315 may implement any type of robotic transformer architecture configured to convert embeddings into action tokens for control of a robotic system. One such suitable architecture is the RT-2 architecture by DeepMind, but other RT model architectures are envisioned. The RT model 315 is configured to process the embedded data from the embedding stage 310 to generate action tokens 320 for controlling the instruments, auxiliary devices, and/or repositionable structures to perform the selected tasks. The action tokens 320 may be textual descriptions of robotic actions that the selected arm, instrument, and/or auxiliary device are to perform to implement the selected task. It should be appreciated that the particular tokens supported by the manipulator system may vary between models. Accordingly, the system 200 may be coupled to a library of RT models 315 each fine-tuned to generate action tokens supported by a different manipulator system type. As a result, the particular outputs of the tuned RT model 315 are adapted to the actual manipulator system associated with the system 200.

The robotic action module 250 then inputs the action tokens 320 to a robotic controller 325 that de-tokenizes the action tokens 320 into actual control commands the control operation of the selected instruments, arms, and/or auxiliary devices. In examples, to perform a given task, a de-tokenized action token may include a plurality of commands including movements of one or more repositionable structures, control of an action of an instrument (e.g., power up, power down, inject, extract, incise, etc.), control of an auxiliary structure (e.g., surgical bed, display device, etc.), or another command for performing a given task. In some embodiments, the de-tokenized control commands are signals suitable for various types of robotic control architectures that may be implemented at the manipulator system. For example, the de-tokenized control commands may be a signal input into a proportional-integral-derivative (PID) controller that controls a particular joint, or a model predictive control (MPC) signal, and/or other types of control signals. As described further herein, the de-tokenized commands may be specific to a given robotic system or machine. For example, a given system may include manipulators with more degrees of freedom of motion than another system, or an instrument, such as a camera, may have a wider field of view than another camera, and the de-tokenized commands may differ depending on the various capabilities and parameters for a given robotic system.

It should be appreciated that a generic RT model is unaware of the particular instruments, arms, or auxiliary devices coupled to a manipulator system. Accordingly, the outputs of the generic RT model indicate high level actions without regard for the capabilities of the specific robotic system the RT model is controlling. However, in the instant systems, the event data stream 252d includes indications of the coupled instruments, arms, and/or auxiliary devices. Accordingly, the embedding of the event data stream 252d and the tuning process for the RT model 315 enables the RT model 315 to output action tokens that are tailored to the particular manipulator system controller by the RT model 315. As one example, rather than simply outputting a token to, say, cauterize a target anatomical feature, the output token can indicate that the selected instrument is to move along a given trajectory to reach a pose and enable a heating element of the end effector. Additionally, the tuning process enables the RT model 315 to understand the range of motion and other control limits associated with the instruments, arms, and/or auxiliary devices. Thus, by having tokens that are specific to the selected instrument, arm, and/or auxiliary device, the system 200 is able to generate action tokens that better reflect the current state of the manipulator system such that the de-tokenized control commands are more likely to achieve successful task performance, thereby improving the efficiency of the closed-loop control system facilitated by RT models.

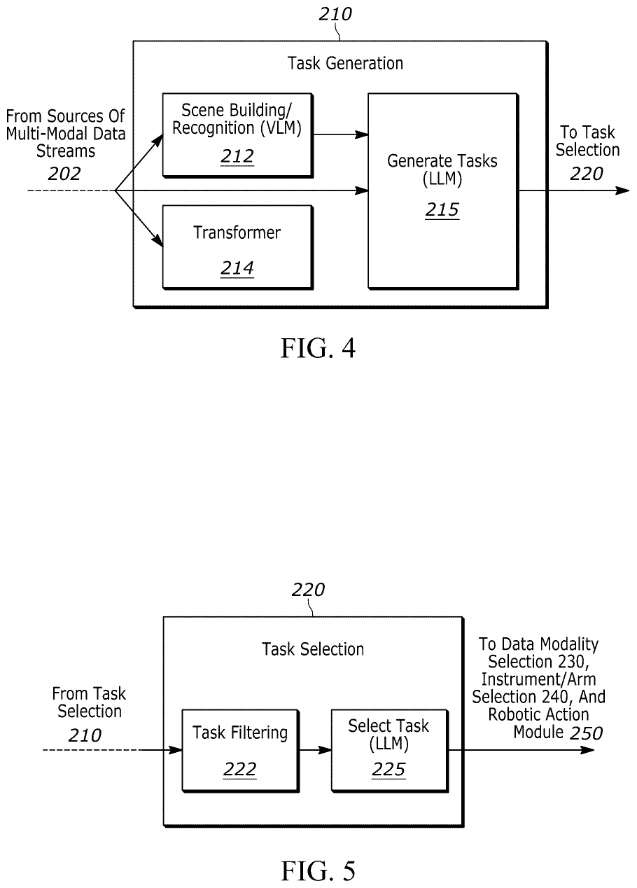

FIG. 4 is a schematic diagram of the task generation module 210 for determining tasks that can be performed based on a current state of the manipulator system and/or procedure as reflected by the multimodal data streams 202. As shown in FIG. 2, the task generation module 210 is configured to receive the multi-modal data streams 202 as an input. Similar to the embedding stage 310 of the robotic action module 250, the task generation module 210 may embed the data streams 202 for input into a task generation model 215. In some embodiments, the embedding models used to embed the data streams 202 are the same as the embedding models included in the embedding stage 310 of the robotic action module 250.

For example, the embedding model for an endoscopic data stream may be routed to a vision-language model (VLM) such as scene recognition model 212. The VLM may be configured to output descriptions of a scene depicted by the image data output by an endoscopic instrument. In surgical applications, for example, the VLM may be configured to identify objects, such as surgical instruments and devices (e.g., surgical beds or tables, medical devices, display devices, etc.), and/or individuals (e.g., clinicians, doctors, medical technicians, etc.) depicted by an image data stream. In examples where the image data is of the operating room, the VLM may be configured to identify surgical activity being performed by the individuals.

As another example, the embedding model of the event data stream may be configured to analyze the time series event data using a transformer model 214 to generate an output token identifying a particular phase of the procedure. It should be appreciated that while the term “transformer model” is used herein, in other embodiments, other model architectures suitable for analyzing temporal dependencies may be implemented. These output tokens may be input into the task generation model 215 to generate tasks that are in furtherance of operation typically associated with the identified phase. Similarly, the transformer model 214 may be configured to detect the completion of a phase or sub-phase of the procedure and generate an output token identifying the transition. These output tokens may be input into the task generation model 215 to begin generating tasks to implement actions associated with a subsequent phase or subphase. Additionally, the transformer model 214 may be configured to detect anomalous operation associated with phase of the procedure and output token indicative of the anomaly (e.g., a token indicative that anomalous operation is occurring, a token indicating a particular type of anomaly that is occurring, and so on). These output tokens may be input into the task generation model 215 to generate tasks to correct the anomaly.

In additional to generating tokens based on time-dependencies in the event data stream, the transformer model 214 may also be configured to output tokens based on an instantaneous representation in the event data stream. To this end, the event data in the event data stream may indicate the complete state of the robotic components of the manipulator system at any given time. Accordingly, the transformer model 214 may be configured to identify equipment operatively coupled to the control system (e.g., manipulator arms, instruments, auxiliary devices) to generate an inventory of devices. In some embodiments, the inventory may also be input to the task generation model 215 such that the task generation model 215 understands that capabilities of the control system and generates tasks that can be implemented thereby.

In addition to identifying equipment currently coupled to the control system, an embedding model for another data stream may be configured to detect equipment that is otherwise available for use in the procedure (e.g., sterilized equipment on-hand in an operating room, equipment available elsewhere at the site). For example, the on-hand equipment may be detected via an operating room video data stream or via an equipment scheduling data stream.

The tokens generated by the embedding models may then be input into the task generation model 215. The task generation may be a LMM configured to directly accept image data streams or an LLM that accepts the natural language descriptions of the image data streams. The task generation model 215 may then generate tasks in furtherance of the detected (or subsequent) phase of the procedure based on the available equipment, personnel, and any other conditions indicated by the input data streams 202.

It should be appreciated that the task generation model 215 may be configured to output multiple tasks to implement the actions associated with a particular phase of the procedure. For example, to implement a procedural phase associated with advancing the instruments to a worksite, the task generation model 215 may generate tasks associated with aligning the instruments with respective ports and advancing the instruments towards the worksite. It should be appreciated that the task generation model may be configured to generate multiple different sets of tasks for implementing the procedural phase. To this end, the different sets of tasks may be performed to implement a procedural phase. Similarly, the tasks within a set of tasks may be performed in different sequences. This enables the various different approaches to performing a procedural phase to be evaluated such that an optimal set of tasks is ultimately selected for implementation.

FIG. 5 is a schematic diagram of the task selection module 220 configured to select one or more tasks and/or sets of tasks generated by the task generation module 210 for implementation by the control system. As illustrated, the task selection module 220 may select the tasks in two stages.

In the first stage, a task filtering model 222 is implemented to categorize the generated tasks for filtering. For example, the task filtering model 222 may categorize each task is capable of being performed autonomously, semi-autonomously, manually, or that the task cannot be performed. Accordingly, tasks that are not capable of being performed may be filtered out. It should be appreciated that while the task generation model 215 is generally configured to generate tasks in view of the configuration of the manipulator system, the generative nature of LLMs and LMMs may still result in the generation of tasks that cannot be performed. Accordingly, the task filtering model 222 may function as a sanity check mechanism on the task generation model 215 to ensure only feasible tasks are implemented.

In a second stage, the remaining tasks are input into a task selection model 225. In some embodiments, the task selection model 225 is an LLM. The task selection model 225 is configured to analyze the potential tasks or sets of tasks to be performed and select a preferred task or set of tasks to implement. For example, the task selection model 225 may be configured to evaluate the tasks or set of tasks using one or more evaluation metrics (e.g., time to perform the task, confidence in ability to autonomously perform the task, an ease of manual implementation, a confidence in safe performance of the task, or relevant tasks).

The task selection model 220 then outputs the selected task(s) and provides the selected task(s) to the data modality selection model 230, instrument/arm/auxiliary device selection module 240, and the robotic action module 250.

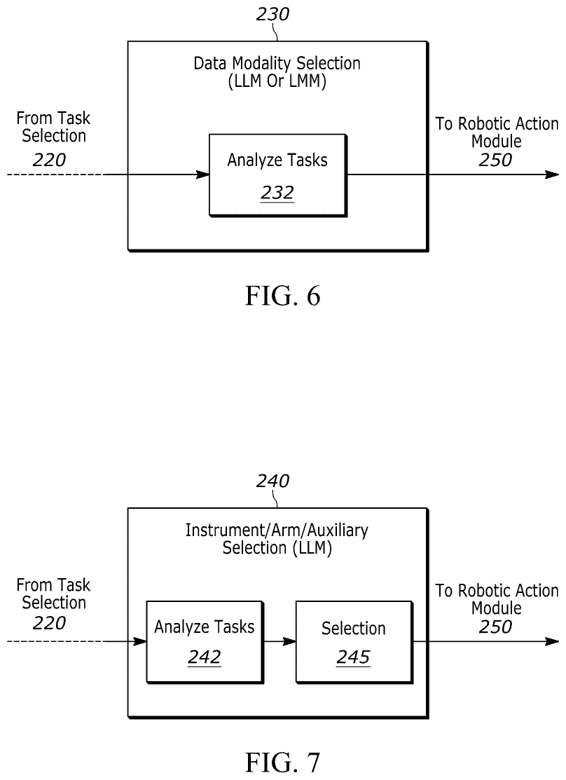

FIG. 6 is a schematic diagram of an example data modality selection module 230 of FIG. 2 for determining task specific data streams for controlling a repositionable structure (such as the repositionable structures 120 of FIG. 1) to perform a task (such as the tasks selected by the task selection module 220). Accordingly, the data modality selection module 230 may be communicative coupled to the task selection module 220.

As illustrated, the data modality selection module 230 may include a task analysis model 232 configured to identify the data modalities required to perform an input task. The task specific data modalities may include one or more streams of data including, without limitation, one or more of image, video, audio, force feedback, user input, event, or instrument provided data streams. In some embodiments, the task analysis model is an LLM. In these embodiments, the task analysis model 232 may be configured to generate a prompt to the LLM that asks the LLM to identify the data streams required to perform the input task. In some embodiments, the input includes a description of the available multi-modal data streams 202 and/or a state thereof. Additionally, the input may include a description of additional data streams that are not currently available and/or alternative states of the available data streams that may be achieved.

Based on the inputs, the task analysis model 232 may be configured to output a set of data modalities required to perform implement the task and associate the input task with the corresponding set of data modalities. For example, the task analysis model 232 may identify a type of task associated with the input task (e.g., a repositioning task, an end effector activation task, a configuration task, etc.) to identify which data modalities are required to perform the input task such that non-necessary data modalities can be ignored when implementing the task thereby providing the above-described improvements in execution time by the robotic action module 250. For example, endoscopic image data may not be required to perform a task related to mounting a new instrument to a robotic arm. As another example, operating room image data may not be required to ablate a target anatomy. As yet another example, a task to change the state of an auxiliary device may not require kinematic data associated with manipulator system. In some embodiments, the data modality selection model 230 includes a set of rules that input into the task analysis model 232 to instruct the task analysis model 232 on how to perform the analysis.

If the required data modalities are available, the data modality selection model 230 may then output the task and data modalities to the robotic action module 250 for implementation at the appropriate time. In the required data modalities are not available, the data modality selection model 230 may generate an output to the task generation module 210 instructing the task generation module 210 of the need to make the additional data modalities available. In some embodiments, the output may be a plain text instruction (e.g., “enable endoscope video feed,” “couple endoscope to instrument X,” or “move endoscope to include view of target anatomy”). In response, the task generation module 210 may generate the instructed tasks to autonomously (or semi-autonomously) make the indicated data modalities available. It should be appreciated that the data modality selection module 230 or the robotic action module 250 may queue the originally input task until detecting that the task to make the required data modality available has been successfully performed. For example, the system 200 may generate an alert to a user to a surgeon or other personnel that a force sensing instrument is needed to perform a suturing task and await the detection of an event indicating the coupling and/or positioning of the force sensing instrument prior to performing the suturing task.

FIG. 7 is a schematic diagram of an example instrument/arm/auxiliary device selection module 240 for assigning instruments and/or arms for performing specific tasks. The instrument/arm/auxiliary device selection module 240 receives the selected tasks from the task selection module 220.

As illustrated, the instrument/arm/auxiliary device selection module 240 may first implement analyze the input task using the task analysis model 242 to identify which instruments/arms are capable of implementing the input task. In some embodiments, the task analysis model 242 is an LLM. In some embodiments, the task analysis model 242 may detect an identifier of the arms, instruments in an event data stream to generate a list of current equipment coupled to the control system. In other embodiments, the robotic action module 250 may generate the list of current equipment and communicate and changes to the instrument/arm/auxiliary device selection module 240. Regardless, the instrument/arm/auxiliary device selection module 240 may then input the list of equipment to the task analysis model 242 along with the input task to generate a list of instruments, arms, or auxiliary devices capable of implementing the task.

The instrument/arm/auxiliary device selection module 240 may then input the list of capable instruments, arm, or auxiliary device into a selection model 245 to assign the task to a particular instrument, arm, or auxiliary device. In some embodiments, the selection model 245 is also an LLM. Accordingly, in these embodiments, the models 242 and 245 may be combined into a single model and the instrument/arm/auxiliary device selection module 240 is configured to generate an input that implements the functionality described with respect to each of the models 242, 245.

The selection model 245 may be configured to select the instrument, arm, or auxiliary device based on several factors. For example, the selection model 245 may analyze kinematic data indicative of the pose of the capable instruments, arms, and auxiliary devices and project a motion that would implement the task. The selection model 245 may then analyze the projected motion with respect to one or more factors (e.g., time to implement, ease of execution, proximity to sensitive regions, force exerted on equipment, range of motion, etc.). In some embodiments, the selection model 245 may also accept indications of user or facility preferences as an input. For example, a left-handed operator may prefer to perform certain actions via the arm or instrument coupled to a user input device held in the operator's left hand. As another example, a highly-skilled operator may prefer to prioritize time of implementation more than a trainee operator.

In some embodiments, if the task involves semi-autonomous or manual implementation, the instrument/arm/auxiliary device selection module 240 may output the assignments to a display device for operator approval. If the operator disapproves the task assignment, the selection model 245 may generate a new proposed assignment or select a lower ranked assignment option not previously present to the operator. Alternatively, the operator may provide a user indication of a preferred assignment. Regardless, after the instrument/arm/auxiliary device selection module 240 determines the final assignment of task to instrument, arm, or auxiliary device, the instrument/arm/auxiliary device selection module 240 may output the assignments to the robotic action module 250 for implementation thereat. In embodiments where the control system includes both the data modality selection module 230 and the instrument/arm/auxiliary device selection module 240, the robotic action module 250 may be configured to associate the outputs of the modules 230, 240 corresponding to the same task such that action is implemented in the manner prescribed by both modules 230, 240.

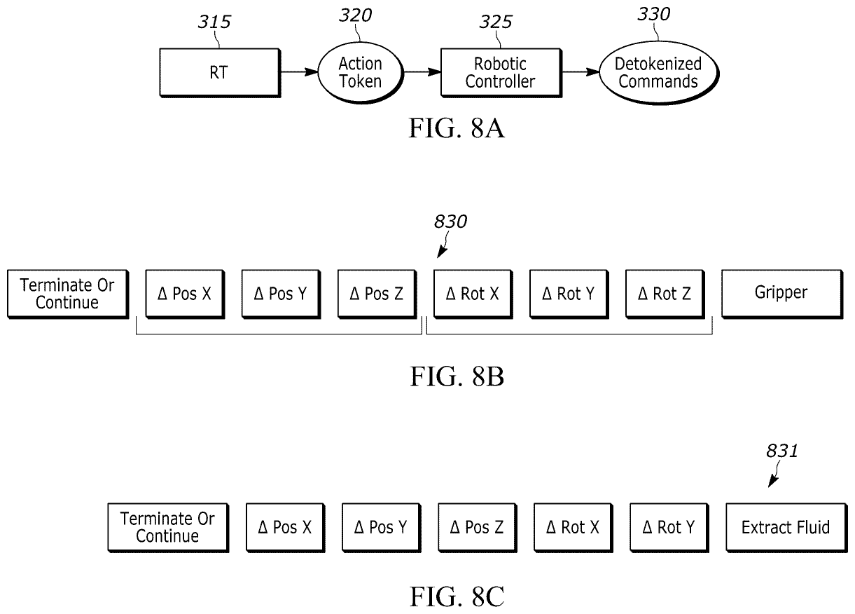

FIG. 8A is a schematic diagram illustrating detokenizing action tokens to control a repositionable structure (such as the repositionable structures 120 of FIG. 1), an instrument, or an auxiliary device. Detokenization is a process under which a system converts an action token 320 (e.g., a text string or commands) output by the robotics transformer model 315 into actual robotic commands 330 to control the indicated equipment, for example, by changing the pose or by activating or otherwise controlling a functionality supported by the indicated equipment.

As described above, the RT model 315 may be fine-tuned and/or selected based on the particular manipulator system model. As such, knowledge of the capabilities of the manipulator system are incorporated into the RT model 315 itself. Similarly, the RT model 315 may be configured to maintain a list of current equipment coupled to the control system. Accordingly, the action tokens 320 output by the RT model 315 may include a textual indication of the equipment that is to perform the action. For example, an output action token 320 may state “Move Instrument X coupled to Arm A to target work area” or “Use Instrument Y to cauterize incision.” Accordingly, the action tokens 320 and detokenization are specific to a robotic system or model based on the capabilities of a given robotic system.

In some embodiments, the action tokens 320 may be further tailored to the current state of the manipulator system. For example, the RT model 315 may be configured to accept a kinematic data stream as an input such that the output action tokens indicate a pose for the instrument and/or arm in the robotic coordinate system. Accordingly, as one example, rather than outputting an action token 320 indicating that an instrument should move to the target work area, the output action token 320 may instead indicate “Move Instrument X to position (x, y, z) and orientation (α, β, γ).” As a result, the action tokens 320 output by the RT model 315 are tailored to the specific state of the manipulator system improving the ability of the control system to accurately interpret and implement autonomously-generated control commands.

Similarly, because the RT model 315 is aware of the functionality supported by the instruments coupled to the control system, the RT model 315 is able to output commands that are specifically tailored to the implementing instrument. For example, rather than outputting an action token that states “Clamp blood vessel,” the RT model 315 may instead output an action token 320 that states “Place gripper around blood vessel at position (x, y, z) and engage grip to exert 50 pascals of force.” As a result, the RT model 315 is able to generate action tokens 320 that have improved alignment with the actual functionality supported by the control system, thereby enabling more precision in the generation of the action tokens 320 and more robust usage of instrument functionality.

After the RT model 315 generates the action tokens 320, the action tokens 320 are input into the robotic controller 325 to convert the natural language action tokens into de-tokenized commands 330 (e.g., parameterized commands used by the control system actually realize the instructed actions). With simultaneous reference to FIGS. 8B and 8C, depicted are example parameterized command structures utilized by the control system.

The de-tokenized command 830 of FIG. 8B is configured to control operation of a gripper instrument that has 6 degrees of freedom (DOFs). Accordingly, the de-tokenized command 830 may include deltas by which the control system is to change each DOF.

Additionally, the de-tokenized command 830 includes additional DOFs related to the functionality supported by the instrument. For example, in the illustrated example related to a gripper, the “gripper” DOF may indicate an amount of force the gripper should exert. Similarly, the de-tokenized command 831 of FIG. 8C is configured to control operation of a fluid extraction instrument that has 5 motive degrees of freedom (DOFs) and one functional DOF. It should be appreciated that the length of the de-tokenized control commands 330 may vary based on the number of motive and functional DOFs supported by the controlled equipment. Accordingly, the length of the de-tokenized commands 330 have fewer unused parameters, thereby enabling more efficient usage of control buses.

In some embodiments, the de-tokenized commands 330 may also control one or more user feedback devices, such as a display device. For example, a de-tokenized command 330 may be configured to cause a display unit to provide an instruction for executing a task that is to be performed manually or semi-autonomously by a user.

While FIGS. 2-8C describe a process for converting the multimodal data streams 202 into de-tokenized commands 330 to implement a one or more tasks, it should be appreciated that the disclosed process may be repeated throughout the procedure until its completion. As a result, each stage of the procedure may be discretized into tasks, that are converted into de-tokenized commands such that any portion of the procedure can be implemented via closed-loop autonomous control. It should be appreciated that in some embodiments, the modules 210, 220, 230, and/or 240 may analyze the multimodal data stream 202 to anticipate future tasks that are predicted to be performed based on a current state. Thus, the tasks analyzed by the modules 210, 220, 230, and/or 240 may differ from the task being implemented by the robotic action module 250. As a result, the control system is able to anticipate future actions in order to ensure closed-loop control of the overall procedure occurs more efficiently.

In some embodiments, the control system may further include a scheduler module configured to generate a sequence of expected tasks (and their corresponding data modalities and/or assigned arms, instruments, or auxiliary devices) and their corresponding triggers for execution. Accordingly, when the scheduler detects, based on the multimodal data streams 202, that a trigger event has occurred, the scheduler can route one or more subsequent tasks to robotic action module 250. It should be appreciated that if the multimodal data streams 202 indicate that conditions have changed (e.g., an anomalous condition is detected), the scheduler can adjust the priority and/or sequencing to prioritize addressing the current conditions.

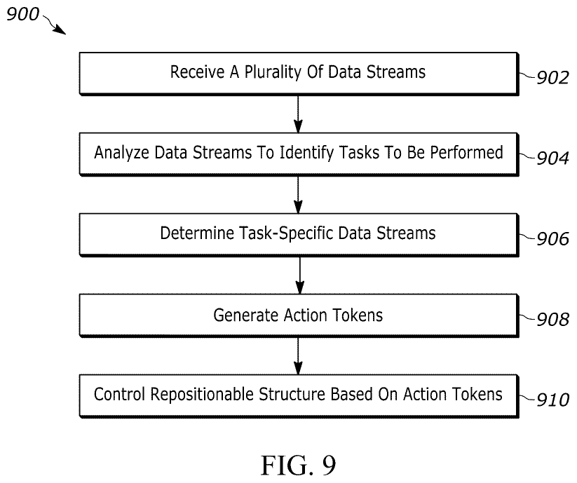

FIG. 9 is a flow diagram of a method 900 for determining task specific data streams for controlling a repositionable structure (such as the repositionable structures 120 of FIG. 1). The method 900 may be performed by a processor system or a control system (such as the processor system 150 and control system 140 of FIG. 1). In implementations, the repositionable structures may be operably coupled to one or more instruments (such as the instruments 122). The one or more instruments may include an endoscope, a camera, a syringe, an incision device, needle driver, scissors, grasping instrument, stapler, etc.

The method 900 may begin at block 902 when the control system receives a plurality of data streams (such as the multimodal data streams 202) from one or more data sources. The data streams may include multi-modal data streams of different types of data, as provided by different devices or systems. For example, the multi-modal data streams may include data from a video camera, audio data, force sensor data, system events (such as the events data stream 252d), endoscopic image data (such as the procedure video data stream 252a), operating room image data, kinematics data (such as the kinematics data stream 252b), haptics data, force data (such as the force data stream 252c), shape sensing data, tissue impedance data, environmental data, and intraoperative image data. Accordingly, the one or more data streams may include environmental data including one or more of data indicative of an interaction between the system and its environment, force data associated with instrument contact with patient tissue, force data associated with feedback to one or more manipulators, and data indicative of system collisions with objects in the environment.

At block 904, the control system may analyze one or more data streams from the plurality of data streams to identify one or more tasks to be performed by one or more repositionable structures, one or more instruments, or one or more auxiliary devices (such as by implementing functionality described with respect to the task generation module 210). The one or more tasks may include changing an energy setting of an instrument or device, controlling an insufflator, controlling a camera, controlling a surgical bed, change a focus or zoom of a camera, change an imaging modality (e.g., from white light to fluorescent light), activating a laser, etc. Additionally, the control system may identify one or more tasks to be performed by an instrument operatively coupled to one or more repositionable structures or by a user in a manual or semi-autonomous manner.

To analyze the kinematic data stream and/or the event data stream, the control system may be configured to implement an embedding model configured to generate a textual description of the kinematic data and/or event data for analysis by the various machine learning models described herein.

To analyze the one or more data streams, the control system may analyze image data streams using a VLM or a VFM (for example using the scene recognition model 212) to generate textual descriptions of the image data for input into a machine learning model (such as the task generation model 215). For example, the VLM or VFM may be configured to perform object recognition and scene building as previously described herein. Accordingly, the control system may be configured to analyze the image data streams to perform one or more of scene perception, object identification, procedure identification, or surgical task detection.

As one example, the control system may provide the event data stream into a transformer model (such as the transformer model 214) trained to identify event or procedural milestones. In this example, the transformer may be further trained to predict future tasks to be performed based on the detection of the event or procedural milestone. In preparation for future tasks to be performed, the control system may further generate one or more tasks to prepare the one or more repositionable structures to perform the predicted future tasks to thereby increasing efficiency of a procedure or operation.

As another example, control system may be configured to detect, via a transformer model, anomalous operation of the computer-assisted system when performing a task and input an indication of the anomalous operation to the modality selection machine learning model to generate a task to diagnose performance of the task. To detect anomalous operation, the control system may detect a shift in sequential predictions beyond a threshold value or an error between embeddings of the actual events and embeddings of the predicted events beyond a threshold. For example, the sequential predictions may determine that a process should take a certain amount of time, and if a task takes longer than the allotted time, the system may identify anomalous operation. In another example, the embeddings of the actual events may provide a data stream indicative of force feedback readings from an instrument with force values much higher, or much lower than predicted embeddings via which the control system may determine anomalous operation.

In examples, the control system may be configured to filter and/or classify the generated tasks to identify whether each task can be performed autonomously, semi-autonomously, manually, or that the task cannot be performed. To filter the tasks, the control system may implement a task selection machine learning model (such as the task filtering model 222). The task selection machine learning model may determine the classification of each task based on available data streams and data stream modalities. In classifying the tasks, the task selection machine learning model may further determine whether a task can be performed autonomously, semi-autonomously, or manually based on the available instruments, or instruments currently installed in one or more repositionable structures.

In some scenarios, the control system may then identify that a task cannot be performed because a required or needed data stream or instrument is not available to perform the respective task. In such scenarios, the control system may generate a new set of tasks or generate a task to enable the needed steam of data.

At block 906, the control system may determine, based on the one or more tasks to be performed and via a modality selection machine learning model (such the task analysis model 232), a set of task-specific data streams. To determine the task-specific data streams, the control system may implement an LLM or an LMM model. In addition to determining task specific data streams, the control system may further determine one or more instruments required to perform a given task, (such as by implementing the instrument/arm/auxiliary device module 240).

At block 908, the control system may generate, based on the set of task-specific data streams and via a robotic action machine learning model (such as the RT 315), one or more action tokens (such as the action tokens 320) for controlling the one or more repositionable structures. In some scenarios, an action token may be configured to cause a display unit to present instructions to a user for performing a manual or semi-autonomous task.

At block 910, the control system may be configured to control the one or more repositionable structures based upon the one or more action tokens. To control the one or more repositionable structures, control system may de-tokenize the action tokens into control commands (such as the de-tokenized commands 330, 830, 831). As previously described, the specific de-tokenized control commands may be specific for a given system, instrument, repositionable structure, or auxiliary device. De-tokenizing the action tokens into control commands may result in control commands with varied lengths depending on the given functionalities and capabilities supported by each individual device and equipment in an environment (e.g., an operating room). For example, the de-tokenized commands may vary in length depending on the number of degrees of freedom (DOFs) and functionalities supported by repositionable structures and/or instruments.

In some embodiments, the control system may determine that a first task has been completed, or is near completion, and embed a set of second task-specific data streams associated with a second task for input into the robotic action machine learning model.

The various machine learning models described herein may be trained using the systems disclosed herein (e.g., the control system 140 and/or the modules thereof). In examples, the machine learning models may be trained using self-supervised learning techniques. For example, a module to process image or video data may be trained using a masked autoencoder learning technique. In this example, after the self-supervised pretraining is completed, the model may then be fine-tuned on historical surgical data in a supervised manner.

In one approach, to train the modality selection machine learning model, the control system may provide a list of all data modalities corresponding the plurality of data streams to the modality selection machine learning model. In response, the modality selection machine learning model may then provide different combinations of data streams to the robotic action machine learning model for various tasks. The robotic action machine learning model may then generate action tokens based on the provided set of task-specific data streams and the corresponding task. In response, the control system may then evaluate task performance for the given task based on the different sets of task specific data streams. The task performances may then be evaluated based on manual labels indicative of how well the control system realized the task. The control system may then use the labels to train the modality selection machine learning model.

In another approach, one or more sets of rules may be developed to identify which types of data modalities are needed for different types of tasks. In this approach, the modality selection machine learning model may be trained to classify an input task as a particular type of task to identify the corresponding set of rules for that task type. Accordingly, in this approach, the control system may use manual labels indicative of task type classification accuracy to train the modality selection machine learning model.

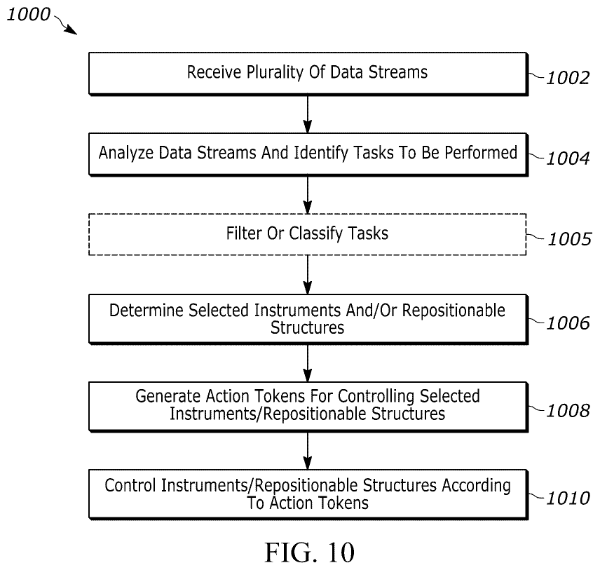

FIG. 10 is a flow diagram of a method 1000 for determining task specific instruments and manipulator arms for controlling a repositionable structure (such as the repositionable structures 120 of FIG. 1). The method 1000 may be performed by a processor system or a control system (such as the processor system 150 and control system 140 of FIG. 1). The method 1000 may begin at block 1002 by receiving a plurality of data streams from one or more data sources. The data streams may include multi-modal data streams of different types of data, as provided by different devices or systems. For example, the data streams may include data from a video camera, medical imaging data (e.g., fluorescent imaging, hyper-spectral imaging, ultrasound imaging, etc.) audio data, force sensor data, system events, endoscopic image data, operating room image data, kinematics data, haptics data, force data, shape sensing data, tissue impedance data, 3D depth data, environmental data, and intraoperative image data.

The method 1000 may further include at block 1004 analyzing one or more data streams from the plurality of data streams to identify one or more tasks to be performed by one or more repositionable structures, one or more instruments, or one or more auxiliaries. To analyze the one or more data streams, the method 1000 may include analyzing image data streams using a VLM or a VFM to generate textual descriptions of the image data for input into a machine learning model. Analyzing the imaging data via the VLM or VFM may further include performing object recognition and scene building as previously described herein. In examples, analyzing the image data streams may include one or more of scene perception, object identification, procedure identification, or surgical task detection. To analyze the one or more data streams, the controller may embed data such as kinematic and/or event data to generate a textual description of the kinematic data and/or event data and the controller may then embed the textual description for further analysis.

In analyzing the one or more data streams, the controller may further provide an event data stream into a transformer model trained to identify event or procedural milestones. In examples, the transformer trained to identify event or procedural milestones may further identify task milestones to predict future tasks to be performed. In preparation for future tasks to be performed, the transformer model may further generate one or more tasks to prepare the one or more repositionable to perform the predicted future tasks to be performed which may streamline the execution of future tasks increasing efficiency of a procedure or operation.

The one or more data streams may include environmental data including one or more of data indicative of an interaction between the system and its environment, force data associated with instrument contact with patient tissue, force data associated with feedback to one or more manipulators, and data indicative of system collisions with objects in the environment. In examples, the one or more data streams may include a set of user preferences.

In some examples, the one or more data streams may include data indicative of the performance of the system or components in performing a task. In such examples, the method 1000 may further include detecting, via a transformer model, anomalous operation of the computer-assisted system or manipulators and/or instruments when performing a task and inputting an indication of the anomalous operation to the actor selection machine learning model to generate a task to diagnose performance of the task. To detect anomalous operation, the controller may detect a shift in sequential predictions beyond a threshold value or an error between embeddings of the actual events and embeddings of the predicted events beyond a threshold. For example, the sequential predictions may determine that a process should take a certain amount of time, and if a task takes longer than the allotted time, the system may identify anomalous operation. In another example, the embeddings of the actual events may provide a data stream indicative of force feedback readings from an instrument with force values much higher, or much lower than predicted embeddings which the system may determine anomalous operation.

In implementations, the repositionable structures may include one or more arms of repositionable structures to perform a task. The one or more instruments may include an endoscope, a camera, a syringe, an incision device, needle driver, scissors, grasping instrument, stapler, etc. In examples, the instruments may include instruments in an operating room, and to determine the selected instrument the controller determines a set of selected instruments in the operating room. The one or more tasks may include changing an energy setting of an instrument or device, controlling an insufflator, controlling a camera, controlling a surgical bed, change a focus or zoom of a camera, change an imaging modality (e.g., from white light to fluorescent light), activating a laser, etc. Additionally, identifying a task to be performed by a repositionable structure may include identifying one or more tasks to be performed by an instrument operatively coupled to one or more repositionable structures. As such, it should be understood that tasks, as described herein, may generally refer to tasks to be performed by a repositionable structure, by an instrument, or by an auxiliary device. Additionally, semi-autonomous and manual tasks may be determined and performed which include input and actions from a user or personnel.

In examples, to determine the tasks, the method 1000 may include an optional block 1005 at which the control system filters or classifies the generated tasks to identify whether each task can be performed autonomously, semi-autonomously, manually, or that the task cannot be performed. To filter the tasks, the method 1000 may use a task selection machine learning model including an LLM. To classify the tasks, the machine learning model may determine the classification of each task based on available data streams, available, repositionable structure arms, and/or the capabilities of the available repositionable structures. In classifying the tasks, the controller may further determine whether a task is to be performed autonomously, semi-autonomously, or manually based on the available instruments, or instruments currently installed in one or more repositionable structures. The controller may classify the tasks using the actor selection machine learning model, or a task selection machine learning model, or another machine learning model different from the actor selection machine learning model. The controller may identify that a task cannot be performed because a required instrument, arm, or ability of an arm (e.g., required degrees of freedom, reach, etc.) is not available to perform the respective task. In such examples, the method may include identifying one or more new tasks and generating a new series of tasks or generating a task to provide the system with a required instrument, or arm, or reposition an arm or instrument to provide the required functionality for performing the task. Further, the controller may determine that a selected or required instrument is not available, and the controller may provide a notification to a user of the missing required instrument via an audio output, display screen, or another device.

The method 1000 further includes at block 1006 determining, based on the one or more tasks to be performed and via a machine learning model, at least one of (i) one or more selected instruments for performing the task, or (ii) a selected repositionable structure of the one or more repositionable structures for performing the task. To determine the selected instruments and/or selected repositionable structures, the method 1000 may include implementing an LLM or an LMM model, such as executed by the data modality selection module 230 of FIGS. 2 and 6. In examples, to determine the selected instruments and/or selected repositionable structures for a set of tasks, the control system may be configured to input indications of a specific task and the available instruments, repositionable arms, and/or capabilities of available repositionable structures into a task selection machine learning model which may include an LLM. In examples, to determine the one or more selected instruments, the control system may evaluate, via the actor selection machine learning model, a set of available instruments for performing one or more determined tasks. Additionally, the controller may determine that a selected or required instrument is available but is not supported for operatively coupling with a particular repositionable structure. In such instances, the controller may further generate a task to swap or exchange the selected instrument to a repositionable structure that supports the instrument and associated functionality of the instrument. In examples, the one or more data streams may include user preferences and identifying the selected repositionable structure or the one or more selected instruments may include evaluating the user preferences to rank the one or more repositionable structures.

The method 1000 further includes at block 1008 generating, based on the task and the at least one of the determined selected instrument or selected repositionable structure and via a robotic action machine learning model, one or more action tokens for controlling the selected instruments and/or selected repositionable structures. In implementations, an action token may include an action that causes a display unit to present instructions to a user for performing a manual or semi-autonomous task. The action tokens are additionally provided to a system for controlling the selected instruments and repositionable structures, which may be a same controller, or may be a different processor or network. In implementations, the robotic action machine learning model may include a robotics transformer model. The method 1000 then includes at block 1010 controlling at least one of the determined selected instruments and/or selected repositionable structures to perform the determined task based upon the one or more generated action tokens. To control the selected instruments and/or repositionable structures, the controller, or a processor of the repositionable structures or another processor, may de-tokenize the action tokens into control commands. As previously described, the specific de-tokenized control commands may be specific for a given system, instrument, repositionable structure, or auxiliary device. De-tokenizing the action tokens into control commands may result in control commands with varied lengths depending on the given functionalities and capabilities supported by each individual device and equipment in an environment (e.g., an operating room). In examples, the de-tokenized commands may vary in length depending on the number of degrees of freedom (DOFs) and functionalities supported by repositionable structures and/or instruments.

In examples, the control system may determine a plurality of tasks to be performed by one or more selected instruments and one or more selected repositionable structures. In such examples, the controller may determine, via a task selection machine learning model, various tasks of the plurality of tasks that may be performed simultaneously, or quasi-simultaneously. The control system may, via the task selection machine learning model, coordinate the various tasks to be performed to improve the overall efficiency of performing the plurality of tasks. Additionally, the control system may determine and coordinate, via the task selection machine learning model, various arms and/or instruments to perform the plurality of tasks simultaneously or quasi-simultaneously. For example, one repositionable structure may control the position and operation of a video camera for providing video feedback of a region of a patient while another repositionable structure is controlled to perform an incision in the region of the patient. Therefore, the two tasks of performing an incision with one repositionable arm using a first instrument, may be coordinated with the task of providing a video data stream via a second instrument operated by a second repositionable structure. Accordingly, various tasks may require more than one repositionable structure to work int tandem and the controller may identify such tasks and select and coordinate instruments and repositionable structure for performing the tasks. Additionally, the controller may determine, via a modality selection learning model, a set of task-specific data streams corresponding to each of the tasks of the one or more tasks to be performed.

As described herein, the method 1000 may be performed using various systems including different robotic system models with different capabilities. As such, the training of the various machine learning models may be fine-tuned using historical data indicative of a given computer-assisted system performing known actions. The fine-tuning of a given system allows for the bulk of the training to be performed independent of the specific skills and capabilities of a system which may then be further fine-tuned when provided to a given system in a set environment. This reduces the time and cost for of training the various machine learning models and AI process for implementing the disclosed methods across various systems and in different environments. In any examples, the method 1000 may include training any of the machine learning models described herein to perform the methods described herein. Additionally, any of the steps of the method 1000 may be performed by a single, or by multiple machine learning models. For example, in an implementation, a one machine learning model may be used to determine one or more selected instruments, and a different machine learning model may be used to determine one or more selected repositionable structures.

The various machine learning models for analyzing data streams to identify tasks to be performed, and determining selected instruments and repositionable structures from the tasks to be performed, and generating action tokens for controlling repositionable structures may be trained using the given systems and controllers disclosed herein (e.g., the systems and controllers of FIGS. 1 and 2). In examples, the machine learning models may be trained using self-supervised learning techniques. For example, a module to process image or video data may be trained using a masked autoencoder learning technique. In this example, after the self-supervised pretraining is completed, the model may then be fine-tuned on historical surgical data in a supervised manner.

In one approach, training the various machine learning models of the method 1000 may include providing a list of all available instruments, repositionable structures, and various capabilities of different repositionable structures and systems to the actor selection machine learning model and the actor selection machine learning model may then provide different combinations of the instruments and repositionable structures to the robotic action machine learning model for a given task. The robotic action machine learning model may then generate action tokens based on the provided instrument and repositionable structures and a processor or controller may then determine a respective task performance for the given task based on the provided set of instruments and repositionable structures. Different combinations of instruments and repositionable structures may then be provided to the robotic action machine learning model and task performance may be evaluated for each combination of provided instruments and repositionable structures. The task performances may then be evaluated and preferred, or optimized, instruments and repositionable structures, and specific combinations of instruments and repositionable structures, may be determined for a plurality of given tasks. Further, different sets of available instruments and/or repositionable structures may be provided to the actor selection machine learning model to further train the actor selection machine learning model given different types of instruments and repositionable structures to broaden overall robustness of the method 1000 to operate in different environments.

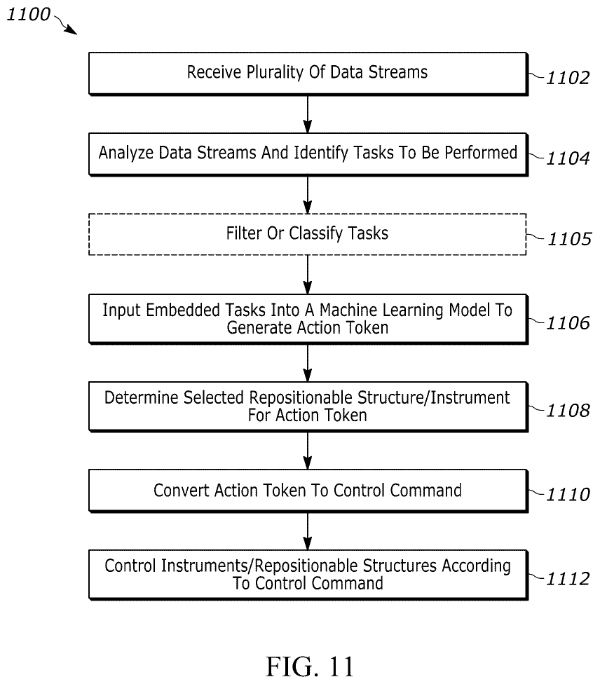

FIG. 11 is a flow diagram of a method 1100 for generating task specific action tokens and determining specific instruments and manipulator arms for controlling a repositionable structure (such as the repositionable structures 120 of FIG. 1). The method 1100 may be performed by a processor system or a control system (such as the processor system 150 and control system 140 of FIG. 1). The method 1100 may begin at block 1102 by receiving a plurality of data streams from one or more data sources. The data streams may include multi-modal data streams of different types of data, as provided by different devices or systems. For example, the data streams may include data from a video camera, medical imaging data (e.g., fluorescent imaging, hyper-spectral imaging, ultrasound imaging, etc.), audio data, force sensor data, system events, endoscopic image data, operating room image data, kinematics data, haptics data, force data, shape sensing data, tissue impedance data, 3D depth data, environmental data, and intraoperative image data.

The method 1100 may further include at block 1104 analyzing one or more data streams from the plurality of data streams to identify one or more tasks to be performed by one or more repositionable structures, one or more instruments, or one or more auxiliaries. To analyze the one or more data streams, the method 1100 may include analyzing image data streams using a VLM or a VFM to generate textual descriptions of the image data for input into a machine learning model. Analyzing the imaging data via the VLM or VFM may further include performing object recognition and scene building as previously described herein. In examples, analyzing the image data streams may include one or more of scene perception, object identification, procedure identification, or surgical task detection. To analyze the one or more data streams, the controller may embed data such as kinematic and/or event data to generate a textual description of the kinematic data and/or event data and the controller may then embed the textual description for further analysis.

In analyzing the one or more data streams, the controller may further provide an event data stream into a transformer model trained to identify event or procedural milestones. In examples, the transformer trained to identify event or procedural milestones may further identify task milestones to predict future tasks to be performed. In preparation for future tasks to be performed, the transformer model may further generate one or more tasks to prepare the one or more repositionable to perform the predicted future tasks to be performed which may streamline the execution of future tasks increasing efficiency of a procedure or operation.

The one or more data streams may include environmental data including one or more of data indicative of an interaction between the system and its environment, force data associated with instrument contact with patient tissue, force data associated with feedback to one or more manipulators, and data indicative of system collisions with objects in the environment. In examples, the one or more data streams may include a set of user preferences.

In some examples, the one or more data streams may include data indicative of the performance of the system or components in performing a task. In such examples, the method 1100 may further include detecting, via a transformer model, anomalous operation of the computer-assisted system or manipulators and/or instruments when performing a task and inputting an indication of the anomalous operation to the actor selection machine learning model to generate a task to diagnose performance of the task. To detect anomalous operation, the controller may detect a shift in sequential predictions beyond a threshold value or an error between embeddings of the actual events and embeddings of the predicted events beyond a threshold. For example, the sequential predictions may determine that a process should take a certain amount of time, and if a task takes longer than the allotted time, the system may identify anomalous operation. In another example, the embeddings of the actual events may provide a data stream indicative of force feedback readings from an instrument with force values much higher, or much lower than predicted embeddings which the system may determine anomalous operation.

In implementations, the repositionable structures may include one or more arms of repositionable structures to perform a task. The one or more instruments may include an endoscope, a camera, a syringe, an incision device, needle driver, scissors, grasping instrument, stapler, etc. In examples, the instruments may include instruments in an operating room, and to determine the selected instrument the controller determines a set of selected instruments in the operating room. The one or more tasks may include changing an energy setting of an instrument or device, controlling an insufflator, controlling a camera, controlling a surgical bed, change a focus or zoom of a camera, change an imaging modality (e.g., from white light to fluorescent light), activating a laser, etc. Additionally, identifying a task to be performed by a repositionable structure may include identifying one or more tasks to be performed by an instrument operatively coupled to one or more repositionable structures. As such, it should be understood that tasks, as described herein, may generally refer to tasks to be performed by a repositionable structure, by an instrument, or by an auxiliary device. Additionally, semi-autonomous and manual tasks may be determined and performed which include input and actions from a user or personnel.

In examples, to determine the tasks, the method 1100 may include an optional block 1105 at which the control system filters or classifies the generated tasks to identify whether each task can be performed autonomously, semi-autonomously, manually, or that the task cannot be performed. To filter the tasks, the method 1100 may use a task selection machine learning model including an LLM. To classify the tasks, the machine learning model may determine the classification of each task based on available data streams, available, repositionable structure arms, and/or the capabilities of the available repositionable structures. In classifying the tasks, the controller may further determine whether a task is to be performed autonomously, semi-autonomously, or manually based on the available instruments, or instruments currently installed in one or more repositionable structures. The controller may classify the tasks using the actor selection machine learning model, or a task selection machine learning model, or another machine learning model different from the actor selection machine learning model. The controller may identify that a task cannot be performed because a required instrument, arm, or ability of an arm (e.g., required degrees of freedom, reach, etc.) is not available to perform the respective task. In such examples, the method may include identifying one or more new tasks and generating a new series of tasks or generating a task to provide the system with a required instrument, or arm, or reposition an arm or instrument to provide the required functionality for performing the task. Further, the controller may determine that a selected or required instrument is not available, and the controller may provide a notification to a user of the missing required instrument via an audio output, display screen, or another device.

The method 1100 further includes at block 1106 embedding one or more tasks and one or more data streams of the plurality of data streams into a robotic action machine learning model to generate one or more action tokens. The generated action tokens are for controlling one or more repositionable structure and/or instruments as described previously herein. The robotic action machine learning model may be a robotics transformer model that receives the embedded tasks and data streams and further generate the one or more action tokens. The action tokens are additionally provided to a system for controlling instruments and repositionable structures, which may be a same controller, or may be a different processor or network.

At block 1108, the method 1100 includes determining a selected repositionable structure or a selected instrument to implement the generated one or more action tokens. In examples, multiple repositionable structures and/or multiple instruments may be selected to implement the one or more action tokens. The control system may utilize one or more machine learning models to determine the selected repositionable structures and/or selected instruments for implement the action tokens. To determine the selected repositionable structures and/or instruments, the method 1100 may include implementing an LLM or an LMM model. In examples, to determine the selected instruments and/or selected repositionable structures to implement one or more action tokens, the control system may be configured to input indications of a specific task and the available instruments, repositionable arms, and/or capabilities of available repositionable structures into a machine learning model which may include an LLM. For example, the control system may determine the one or more selected instruments or selected repositionable structures using the actor selection machine learning model and based on a range of motion, an accessible or reachable volume, an accessible volume without collision, an instrument functionality, or a remaining lifetime of an instrument. In examples, to determine the one or more selected instruments, the control system may evaluate, via the actor selection machine learning model, a set of available instruments for implementing one or more action tokens.

The controller may determine that a selected or required instrument is available but is not supported for operatively coupling with a particular repositionable structure. In such instances, the controller may further generate a task, which may include one or more action tokens, to swap or exchange the selected instrument to a repositionable structure that supports the instrument and associated functionality of the instrument. The control system may further provide a notification to a user of the missing selected instrument. In examples, the one or more data streams may include user preferences and identifying the selected repositionable structure or the one or more selected instruments may include evaluating the user preferences to rank the one or more repositionable structures and/or instruments for implementing the one or more action tokens. The control system may further determine the selected instrument by evaluating a set of available instruments for suitability for performing a task of the one or more tasks.

The method 1100 further includes converting the one or more action tokens to an action sequence that is specifically adapted to the selected repositionable structure and/or instrument. The action sequence includes a series of commands that are specific to a given repositionable structure and/or instrument and respective capabilities of the repositionable structure and/or instrument. Additionally, action sequences will have varied lengths depending on the given functionalities and capabilities supported by each individual device and equipment in an environment (e.g., an operating room). In examples, the action sequence may vary in length depending on the number of degrees of freedom (DOFs) and functionalities supported by repositionable structures and/or instruments. For example, one repositionable structure may be capable of moving in three dimensions, while another repositionable structure may only be able to move in two dimensions. As such, respective action sequences will differ for each of these repositionable structures according to moving the structures in three and two dimensions respectively.

The method 1100 then includes at block 1112 controlling at least one of the determined selected repositionable structure and/or selected instrument to perform the determined task based upon the one or more action sequences. As previously described, the specific de-tokenized control commands in the form of an action sequence are specific for a given system, instrument, repositionable structure, or auxiliary device. The control system may then cause the selected repositionable structure and/or instrument to perform the actions in the action sequence to accomplish one or more of the tasks. Controlling the selected repositionable structure and/or instrument may include de-tokenizing the action tokens into control commands.

As described herein, the method 1100 may be performed using various systems including different robotic system models with different capabilities. As such, the training of the various machine learning models may be fine-tuned using historical data indicative of a given computer-assisted system performing known actions. The fine-tuning of a given system allows for the bulk of the training to be performed independent of the specific skills and capabilities of a system which may then be further fine-tuned when provided to a given system in a set environment. This reduces the time and cost for of training the various machine learning models and AI process for implementing the disclosed methods across various systems and in different environments. In any examples, the method 1100 may include training and of the machine learning models described herein to perform the methods described herein. Additionally, any of the steps of the method 1100 may be performed by a single, or by multiple machine learning models.

One or more components of the examples discussed in this disclosure, such as control system 140, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 502.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain examples and examples have been described above and shown in the accompanying drawings, it is to be understood that such examples and examples are merely illustrative and are not limited to the specific constructions and arrangements shown and described, since various other alternatives, modifications, and equivalents will be appreciated by those with ordinary skill in the art.

What is claimed is:

1. A computer-assisted system, the system comprising:
one or more repositionable structures operatively coupled to one or more instruments; and
a control system operably coupled to the one or more repositionable structures, wherein the control system is configured to:
receive a plurality of data streams from one or more data sources;
analyze one or more data streams from the plurality of data streams to identify a task to be performed by the one or more repositionable structures or the one or more instruments;
determine, based on the task to be performed and via an actor selection machine learning model, at least one of (i) one or more selected instruments for performing the task, or (ii) a selected repositionable structure of the one or more repositionable structures for performing the task;
generate, via a robotic action machine learning model, one or more action tokens for controlling the at least one of the selected instruments or the selected repositionable structures; and
control the at least one of the selected instruments or the selected repositionable structures to perform the task based upon the one or more generated action tokens.

2. The computer-assisted system of claim 1, wherein to determine the one or more selected instruments the control system is further configured to:
determine a set of available instruments in an operating room; and
evaluate, via the actor selection machine learning model, the set of available instruments for suitability for performing the task,
wherein suitability for performing the task is based upon one or more of a range of motion, an accessible or reachable volume, an accessible volume without collision, an instrument functionality, or a remaining lifetime.

3. The computer-assisted system of claim 1, wherein the control system is further configured to:
determine, via the actor selection machine learning model, that a selected instrument is not available; and
provide a notification to a user of instrument unavailability.

4. The computer-assisted system of claim 1, wherein the control system is further configured to:
determine, via the actor selection machine learning model, that a selected instrument is available but not currently supported by the selected repositionable structure; and
generate a task to swap an instrument currently supported by the selected repositionable structure with the selected instrument.

5. The computer-assisted system of claim 1, wherein:
the one or more data streams include a set of user preferences; and
to determine the selected repositionable structures or the selected instruments, the control system is further configured to:
evaluate the user preferences to rank the one or more repositionable structures or instruments.

6. The computer-assisted system of claim 1, wherein to identify the task to be performed, the control system is configured to:
determine, via a task generation machine learning model, a set of tasks to be performed.

7. The computer-assisted system of claim 1, wherein:
to identify the task to be performed, the control system is configured to determine, via a task generation machine learning model, a set of tasks to be performed; and
the control system is further configured to classify, via the actor selection machine learning model and based on the set of tasks, whether each task can be performed autonomously, semi-autonomously, manually, or that the task cannot be performed.

8. The computer-assisted system of claim 1, wherein:
to identify the task to be performed, the control system is configured to determine, via a task generation machine learning model, a set of tasks to be performed; and
the control system is configured to classify, via a task selection machine learning model and based on the set of tasks, whether each task can be performed autonomously, semi-autonomously, manually, or that the task cannot be performed.

9. The computer-assisted system of claim 8, wherein to classify the set of tasks, the control system is configured to:
classify the set of tasks based on at least one of (i) an analysis of the received data streams or (ii) functionality supported by the one or more repositionable structures or the one or more instruments.

10. The computer-assisted system of claim 1, wherein:
to identify the task to be performed, the control system is configured to determine, via a task generation machine learning model, a set of tasks to be performed; and
the control system is further configured to determine, via a modality selection machine learning model, a set of task-specific data streams corresponding to each of the tasks of the set of tasks to be performed.

11. The computer-assisted system of claim 1, wherein the plurality of data streams includes one or more of system events, endoscopic image data, operating room image data, kinematics data, haptics data, force data, shape sensing data, tissue impedance data, environmental data, and intraoperative imaging.

12. The computer-assisted system of claim 11, wherein the control system is further configured to:
analyze image data streams using a visional-language model (VLM) or a vision-foundation model (VFM) to generate textual annotations of the image data; and
input the textual annotations into the task generation machine learning model,
wherein the textual annotations include at least one of scene perception, object identification, procedure identification, or surgical task detection.

13. The computer-assisted system of claim 11, wherein the control system is further configured to:
analyze the kinematic data stream and/or the event data stream to generate a textual description of the kinematic data stream and/or the event data stream; and
input the textual description into the task generation machine learning model.

14. The computer-assisted system of claim 11, wherein the control system is further configured to:
input the system events data stream into a transformer model trained to analyze time-series event data to predict a state associated with operation of the computer-assisted system; and
input the predicted state into the task generation machine learning model,
wherein the state associated with operation of the computer-assisted system comprises at least one of a current step of a procedure, a predicted future step of the procedure, anomalous operation during the procedure, or an amount of life remaining for an instrument.

15. The computer-assisted system of claim 1, wherein to control the at least one of the determined selected instruments or selected repositionable structures the control system is configured to:
de-tokenize the one or more action tokens into control commands.

16. A method for performing automated surgical tasks via a computer-assisted system comprising one or more repositionable structures operatively coupled to respective instruments, and a control system operatively coupled to the one or more repositionable structures, the method comprising:
receiving a plurality of data streams from one or more data sources;
analyzing the data streams to identify a task to be performed by the one or more repositionable structures;
determining, based on the task to be performed and via an actor selection machine learning model, at least one of (i) one or more selected instruments for performing the task, and (ii) one or more selected repositionable structures of the one or more repositionable structures for performing the task;
generating, via a robotic action machine learning model, one or more action tokens for controlling the at least one of the determined selected instruments or the selected repositionable structures; and
controlling the at least one of the determined selected instruments or selected repositionable structures to perform the task based upon the generated action tokens.

17. A computer-assisted system for performing automated tasks, the system comprising:
one or more repositionable structures configured operatively coupled to respective instruments; and
a control system operably coupled to the repositionable structure, wherein the control system is configured to:
receive a plurality of data streams from one or more data sources;
analyze one or more data streams from the plurality of data streams to identify one or more tasks to be performed by the one or more repositionable structures;
input embeddings of the one or more tasks and at least one of the plurality of data streams into a robotic action machine learning model to generate one or more action tokens for controlling the one or more repositionable structures;
determine a selected repositionable structure or selected instrument to implement the action token;
convert the action token to a control command adapted to the selected repositionable structure or instrument; and
control the selected repositionable structure or instrument based upon the control command.

18. The computer-assisted system of claim 17, wherein control commands vary in length depending on at least one of (i) functionalities supported by equipment in an operating room, or (ii) degrees of freedom supported by the selected repositionable structure or instrument.

19. The computer-assisted system of claim 17, wherein to determine the one or more selected instruments the control system is further configured to:
determine a set of available instruments in an operating room; and
evaluate, via an actor selection machine learning model, the set of available instruments for suitability for performing the one or more tasks.

20. The computer-assisted system of claim 17, wherein suitability for performing the task is based upon one or more of a range of motion, an accessible or reachable volume, an accessible volume without collision, an instrument functionality, or a remaining lifetime.
